(19) United States
(12) Patent Application Publication
(10) Pub. No.: US 2017/0354552 A1
(43) Pub. Date: Dec. 14, 2017

(54) Tampon E-Z Clean Disposal

(71) Applicant: Angela LaRosa, Lynbrook, NY (US)

(72) Inventor: Angela LaRosa, Lynbrook, NY (US)

(21) Appl. No.: 15/622,683

(22) Filed: Jun. 14, 2017

Related U.S. Application Data
(60) Provisional application No. 62/350,020, filed on Jun. 14, 2016.

(51) Int. Cl. A61F 13/551; A61F 13/20; A61F 13/84

(52) U.S. Cl. CPC A61F 13/5518; A61F 13/8405; A61F 13/20; A61F 2013/8408

(57) ABSTRACT

A hygiene protective pouch comprising: a main panel including a front side, and back side including a plurality of outer edge portions; and secondary panel member. One of the plurality of outer edge portions includes an adhesive thereon adapted such that the main panel can be folded upon itself forming an interior volume that becomes sealed via said adhesive. secondary panel member is smaller in size than said main panel and is includes a plurality of outer edge portions connected to said main portion forming a pocket having an interior volume. The hygiene protective pouch is adapted to receive a soiled hygienic item within the interior volume of the pocket, folded upon itself, and sealed via the adhesive for disposal thereof. A combination is also disclosed of a tampon and a hygiene protective pouch, the combination comprising a tampon and a hygiene protective pouch.

### CROSS-REFERENCE TO RELATED APPLICATION

The present application is related to and claims priority from prior provisional application Ser. No. 62/350,020, filed Jun. 14, 2016 which application is incorporated herein by reference.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

### 1. Field of the Invention

The present invention relates generally to the field of pouch devices and more specifically relates to a hygiene protective pouch structured and arranged to hold and seal a tampon within after use to provide women with a simple, sanitary, and discrete method of disposing of a tampon, without any lingering odor or mess, when visiting the ladies' room.

### 2. Description of the Related Art

A tampon is a mass of absorbent material, primarily used as a feminine hygiene product. Historically, the word “tampon” originated from the medieval French word “tampion”, meaning a piece of cloth to stop a hole, a stamp, plug, or stopper. At present, tampons are designed to be easily inserted into the vagina during menstruation and absorb the menstrual flow. Several countries regulate tampons as medical devices. In the United States, they are considered to be a Class II medical device by the Food and Drug Administration (FDA). They are sometimes used for hemostasis in surgery.

Various attempts have been made to solve problems found in pouch device art. Among these are found in: U.S. Pat. No. 20100094238 to Anthony J. Scarano; U.S. Pub. No. 20070083179 to Amy Fuentes; and U.S. Pat. No. 20070038200 to Krulevitch et al. This prior art is representative of a disposable-hygiene protective pouches.

Ideally, a hygiene protective pouch (also referred to herein as Tampon E-Z Clean Disposal) should be user-friendly and safe in-use and, yet should operate reliably and be manufactured at a modest expense. Thus, a need exists for a hygiene protective pouch structured and arranged to hold and seal a tampon within after use to provide women with a simple, sanitary, and discrete method of disposing of a tampon, without any lingering odor or mess, when visiting the ladies' room and to avoid the above-mentioned problems

### BRIEF SUMMARY OF THE INVENTION

In view of the foregoing disadvantages inherent in the known pouch devices art, the present invention provides a hygiene protective pouch (also referred to herein as Tampon E-Z Clean Disposal). The general purpose of the present invention, which will be described subsequently in greater detail is to provide a hygiene protective pouch structured and arranged to hold and seal a tampon within after use to provide women with a simple, sanitary, and discrete method of disposing of a tampon, without any lingering odor or mess, when visiting the ladies' room.

A hygiene protective pouch is disclosed herein comprising: a main panel and a secondary panel member. The main panel includes a front side, and back side including a plurality of outer edge portions. One of the plurality of outer edge portions includes an adhesive thereon adapted such that the main panel can be folded upon itself forming an interior volume that becomes sealed via said adhesive. The secondary panel member is smaller in size than said main panel and is includes a plurality of outer edge portions connected to said main portion forming a pocket having an interior volume. The hygiene protective pouch is adapted to receive a soiled hygienic item within the interior volume of the pocket, folded upon itself, and sealed via the adhesive for disposal thereof.

A combination is also disclosed of a tampon and a hygiene protective pouch, the combination comprising a tampon and a hygiene protective pouch.

The present invention holds significant improvements and serves as a Tampon E-Z Clean Disposal. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present invention, hygiene protective pouch (also referred to herein as Tampon E-Z Clean Disposal), constructed and operative according to the teachings of the present invention.

FIG. 1 is a perspective view illustrating a hygiene protective pouch according to an embodiment of the present invention of FIG. 1.

FIG. 2 shows a perspective view illustrating a combination of a tampon and a hygiene protective pouch according to an embodiment of the present invention.

FIG. 3 is another perspective view illustrating the combination of a tampon and a hygiene protective pouch according to an embodiment of the present invention of FIG. 1.

FIG. 4 is a perspective view illustrating the combination of a tampon and a hygiene protective pouch according to an embodiment of the present invention of FIG. 1.

FIG. 5 is a perspective view illustrating hygiene protective pouch in a sealed condition with a soiled tampon inside according to an embodiment of the present invention of FIG. 1.

The various embodiments of the present invention will hereinafter be described in conjunction with the appended drawings, wherein like designations denote like elements.

### DETAILED DESCRIPTION

As discussed above, embodiments of the present invention relate to pouch devices and more specifically relates to a hygiene protective pouch (also referred to herein as Tampon E-Z Clean Disposal) structured and arranged to hold and seal a tampon within after use to provide women with a simple, sanitary, and discrete method of disposing of a tampon, without any lingering odor or mess, when visiting the ladies' room.

Generally speaking, the Tampon E-Z Clean Disposal comprising a novel product offering consumers a practical solution to the aforementioned challenges. As the name implies, the Tampon E-Z Clean Disposal comprises a specially designed easily foldable pouch featuring an amply sized pocket for inserting a soiled tampon. The pocket itself and the inner lining of the Tampon E-Z Clean Disposal could be fabricated of a cotton or paper-like material. The outer liner and three of the edges will feature a thin waterproof material to prevent any leakage and to prevent contact from germs or bacteria. The fourth edge would feature tape or an adhesive, to fold and seal the Tampon E-Z Clean Disposal shut for easy transport and disposal. Scented and unscented versions would be produced, with a plethora of fragrances to choose from.

The Tampon E-Z Clean Disposal would provide a clean and sanitary way in which to dispose of a used tampon. Featuring a waterproof outer liner, one would simply drop the tampon into the designated inner pocket, fold in the edges, and seal in the tampon using the adhesive tape edge. Then, simply walk to the trash without worry of leakage onto one's hands, or unpleasant odors. A far better alternative to wrapping a tampon in tissue, which can leak through to the hands and spread germs, the Tampon E-Z Clean Disposal would ensure that one's hands stay clean and dry. As previously mentioned, this scented product would deter pets from rummaging through trash. In addition, by keeping the disposal process discrete and clean, it would also deter users from flushing a tampon down the toilet in an attempt to avoid a messy disposal. And, as we all know, this can cause extensive damage to plumbing, resulting in costly repairs. Providing female consumers a clean and discrete solution to these challenges, the Tampon E-Z Clean Disposal should surely receive a wide and enthusiastic market reception.

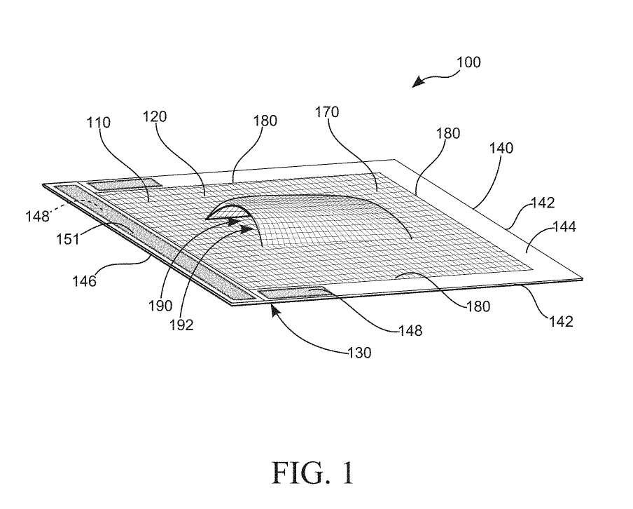

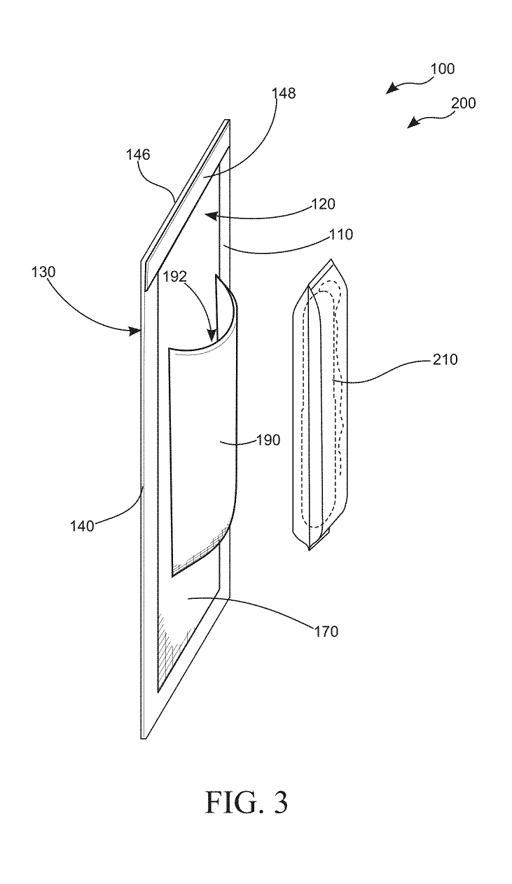

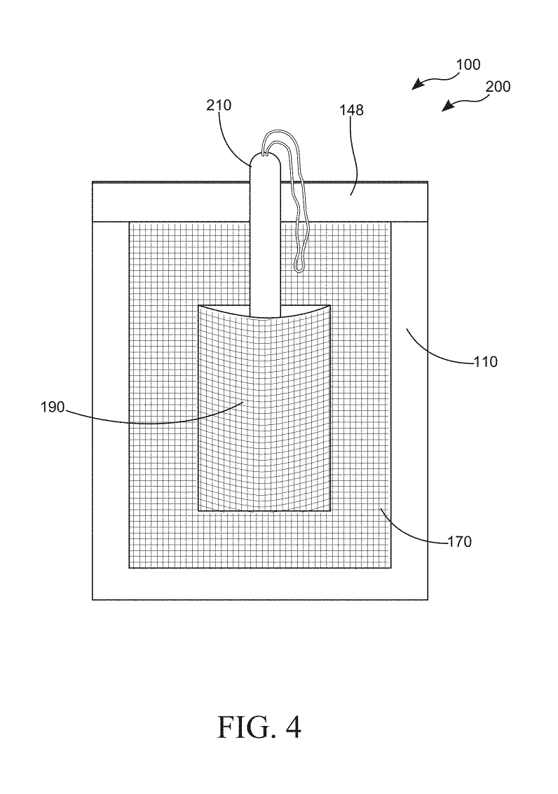

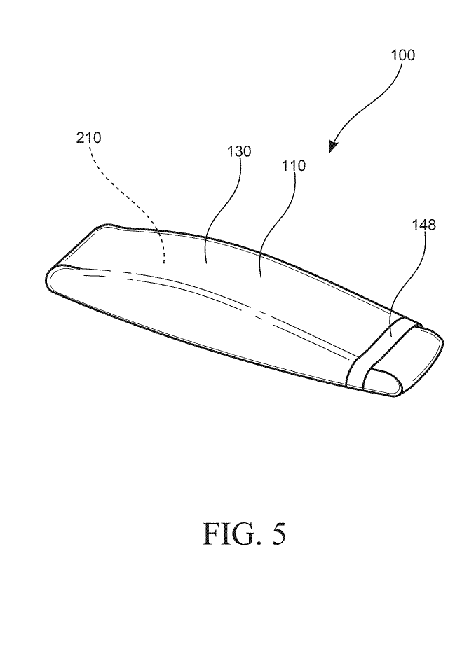

Referring now to FIGS. 1-5, showing perspective views illustrating hygiene protective pouch 100 and combination 200 of tampon 210 and hygiene protective pouch 100 according to an embodiment of the present invention of FIG. 1.

Hygiene protective pouch 100 comprising: main panel 110 and secondary panel member 170. Main panel 110 includes front side 120, and back side 130 including plurality of outer edge portions 140. Main panel 110 is formed from a non-permeable material

Main panel 110 is formed having a rectangular shape. The rectangular shape forms four outer edge portions, such that three outer edge portions 142 includes waterproof material 144 thereon, and fourth outer edge portion 146 includes adhesive 148 thereon. Main panel 110 further includes protective strip 150 releasably covering one outer edge portion (fourth outer edge portion 146) having adhesive 148 thereon, and adapted to be peeled off therefrom to expose adhesive 148 such that main panel 110 can be folded upon itself and sealed via adhesive 148. It should be noted that adhesive may be also located on plurality of outer edge portions 140 and is not just limited to fourth outer edge portion 146 as shown in FIG. 1. Main panel 110 further includes a fragrance material embedded therein adapted to overcome other odors that may result from enclosing soiled hygienic item (tampon 210).

Secondary panel 170 member is smaller in size than main panel 110 and is includes plurality of outer edge portions 180 connected to main portion 110 forming pocket 190 having an interior volume 192. Secondary panel member 170 is formed from a cotton material. Hygiene protective pouch 100 is adapted to receive soiled hygienic item (tampon 210) within interior volume 192 of pocket 190, folded upon itself, and sealed via adhesive 148 for disposal thereof.

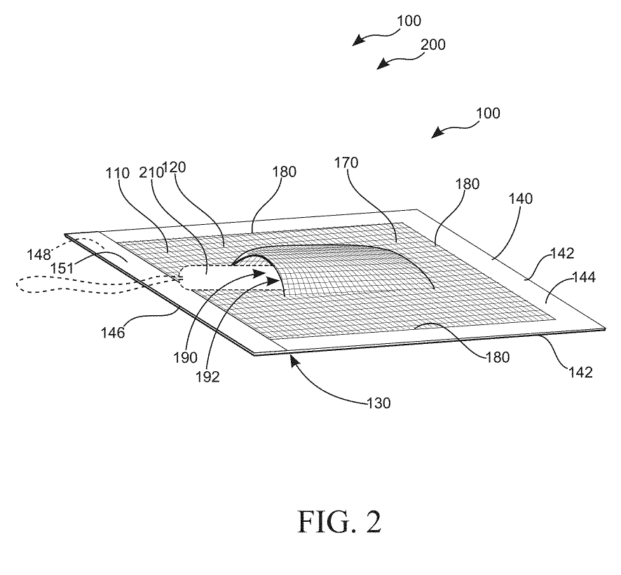

Combination 200 is also disclosed of soiled hygienic item and hygiene protective pouch 100, combination 200 comprising tampon 210 and hygiene protective pouch 100 as shown in FIGS. 2-5.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A hygiene protective pouch comprising:
a main panel including;
a front side;
a back side including;
a plurality of outer edge portions;
wherein one of said plurality of outer edge portions includes an adhesive thereon adapted such that said main panel can be folded upon itself forming an interior volume that becomes sealed via said adhesive;
a secondary panel member;
wherein said secondary panel member is smaller in size than said main panel and is includes a plurality of outer edge portions connected to said main portion forming a pocket having an interior volume;
wherein said hygiene protective pouch is adapted to receive a soiled hygienic item within said interior volume of said pocket, folded upon itself, and sealed via said adhesive for disposal thereof.

2. The hygiene protective pouch of claim 1, wherein said main panel is formed from a non-permeable material.

3. The hygiene protective pouch of claim 2, wherein said secondary panel member is formed from a cotton material.

4. The hygiene protective pouch of claim 2, wherein said main panel is formed having a rectangular shape.

5. The hygiene protective pouch of claim 4, wherein said rectangular shape forms four outer edge portions, such that three said outer edge portions includes a waterproof material thereon, and said fourth outer edge portion includes said adhesive thereon.

6. The hygiene protective pouch of claim 1, wherein said main panel further includes a protective strip releasably covering said one outer edge portion having said adhesive thereon, and adapted to be peeled off therefrom to expose said adhesive such that said main panel can be folded upon itself and sealed via said adhesive.

7. The hygiene protective pouch of claim 1, wherein said main panel further includes a fragrance material embedded therein adapted to overcome other odors that may result from enclosing said soiled hygienic item.

8. A combination of a tampon and a hygiene protective pouch, said combination comprising:
a tampon; and
a hygiene protective pouch comprising:
a main panel including;
a front side;
a back side including;
a plurality of outer edge portions;
wherein one of said plurality of outer edge portions includes an adhesive thereon adapted such that said main panel can be folded upon itself forming an interior volume that becomes sealed via said adhesive;
a secondary panel member;
wherein said secondary panel member is smaller in size than said main panel and is includes a plurality of outer edge portions connected to said main portion forming a pocket having an interior volume;
wherein said hygiene protective pouch is adapted to receive said tampon item within said interior volume of said pocket, folded upon itself, and sealed via said adhesive after use for disposal thereof.

9. The combination of claim 8, wherein said main panel is formed from a non-permeable material.

10. The combination of claim 9, wherein said secondary panel member is formed from a cotton material.

11. The combination of claim 9, wherein said main panel is formed having a rectangular shape.

12. The combination of claim 11, wherein said rectangular shape forms four outer edge portions, such that three said outer edge portions includes a waterproof material thereon, and said fourth outer edge portion includes said adhesive thereon.

13. The combination of claim 8, wherein said main panel further includes a protective strip releasably covering said one outer edge portion having said adhesive thereon, and adapted to be peeled off therefrom to expose said adhesive such that said main panel can be folded upon itself and sealed via said adhesive.

14. The combination of claim 8, wherein said main panel further includes a fragrance material embedded therein adapted to overcome other odors that may result from enclosing said tampon after use.
